(19) United States
(12) Patent Application Publication
(10) Pub. No.: US 2023/0128758 A1
(43) Pub. Date: Apr. 27, 2023

(54) METHODS AND APPARATUS FOR OCULAR EXAMINATION

(71) Applicant: 123 SEE, Inc., Boston, MA (US)

(72) Inventors: Matthias HOFMANN, Wayland, MA (US); Jack MOLDAVE, Boston, MA (US)

(21) Appl. No.: 17/971,240

(22) Filed: Oct. 21, 2022

Related U.S. Application Data
(60) Provisional application No. 63/270,907, filed on Oct. 22, 2021.

(51) Int. Cl. A61B 3/103; A61B 3/11; A61B 3/00; A61B 3/14; A61B 3/18

(52) U.S. Cl. CPC A61B 3/103; A61B 3/112; A61B 3/0025; A61B 3/14; A61B 3/0091; A61B 3/18; A61B 3/0008; A61B 3/0083

(57) ABSTRACT

A system is disclosed for capturing diagnostic eye information. The system includes at least one energy source for directing electromagnetic energy into an eye of a subject, a plurality of perception units, each perception unit being associated with an associated position in the visual field of the eye, and each perception unit being adapted to capture refractive information from the eye responsive to the electromagnetic energy, and a processing system for determining refractive error information associated with each position of each perception unit in the visual field of the eye, and for determining refractive error composite information regarding the eye responsive to the refractive error information associated with each perception unit and independent of a direction of gaze of the eye.

### PRIORITY

The present application claims priority to U.S. Provisional Patent Application No. 63/270,907 filed Oct. 22, 2021, the disclosure of which is hereby incorporated by reference in its entirety.

### BACKGROUND

Automated refractors, or auto-refractors, are instruments designed to quickly measure ocular aberrations, or refractive errors, of the eye. Auto-refractors are commonly used by eye care professionals (ECPs) to assist with determining the eyeglass or contact lens correction numbers of their patients. Historically, auto-refractors were not accurate enough to determine lens correction numbers directly but have found their use by ECPs as a pre-screening tool prior to manual or subjective refraction. Subjective refraction via phoropter remains the tried-and-true method for reaching the final lens correction numbers but is time-consuming, requires an ECP with substantial training, and not all ECPs can accomplish it repeatably and with high accuracy.

The accuracy of conventional auto-refraction techniques based on retinal reflex measurement (optometer, scheiner, retinoscopic, or photo-refractive) is generally limited by different degrees of the following: (a) gaze misalignment with respect to sensor optics, (b) poor control over the eye's accommodative state during measurement, (c) limited ability to detect high order aberrations in the eye, (d) inability to detect medial opacities, and (e) inability to detect eye disease on the anterior or posterior surfaces that affect visual acuity outside of refractive errors. Issues of the retinal reflex techniques are described in more detail below.

The problem of gaze-misalignment is particularly problematic when one camera only is used. In a conventional auto-refractor setup where a single camera sensor is used, it is likely that the position probed on the retina does not coincide with the location of the fovea due to gaze-misalignment of the subject's eye and the sensor's camera. A misaligned eye is essentially rotated in the eye socket pointing some angle away to the camera axis. The fovea is where central vision happens and probing the refractive error outside the fovea, namely the periphery, can cause measurement errors in excess of 0.5 diopters. Several strategies exist to coax the subject's gaze towards the optical center of the camera including displaying visual stimuli and guides, audio prompts, or guidance by the ECP, but these are not guaranteed to work due to involuntary eye movement, difficulty understanding instructions, or being uncomfortable or stressed by the device interface.

The influence of high-order aberrations (HOAs) on normal vision generally depends on the diameter of the pupil. A large pupil diameter (typically in dark settings, e.g. driving at night) increases the influence of HOAs on normal vision since more area of the eye's optics becomes part of image formation. People with significant HOAs (e.g. astigmatism, coma, trefoil) often report “seeing halos” when looking at lights at night. Detecting the full extent of HOAs can be challenging due to changing pupil diameters and the inability to map the entire refractive state of the eye via conventional auto-refraction techniques. Some auto-refraction systems attempt to adjust refraction readings based on pupil diameter and other meta inputs such as age and gender via empirically derived lookup tables, however, this corrects for population averages only.

The eye uses the ciliary muscle to change optical power to focus on near and far objects in what is called accommodation. If the eye is not focused on the desired image plane relative to the sensor camera during measurement, the results may become significantly skewed sometimes in excess of 1.0 diopters. Conventional auto-refractors have no way of determining or validating if the patient is focused on the correct target distance during measurement. Some auto-refractors utilize the fogging technique and set the focal plane of the target image optically beyond infinity, thereby relaxing accommodation during measurement. The fogging is best accomplished in systems where the auto-refractor optics are relatively close to the subjects' eyes which means that the instrument touches the subject's face.

Recent advances in auto-refraction technology have begun to address some of the above issues. The Shack-Hartmann or wavefront measurement techniques are good at determining HOAs and in some cases have been clinically proven to provide more accurate results than subjective refraction by the average ECP with respect to the patients' preferred lens correction values. Up to this point, however, no auto-refraction system has been developed that systematically addresses most of the above outlined issues that lead to limited accuracy.

Additionally, from a usability standpoint auto-refractors were designed to be operated by ECPs in a clinical setting, not by the patient themselves. This effectively forces patients to visit an ECP office to obtain lens correction numbers for new corrective eyewear which is a significant barrier to properly maintained vision care for the average citizen due to exam cost and time availability.

There remains a need therefore, for systems and methods for capturing retinal reflex information of the eye using more efficient and more economical processes, and there remains further a need for such systems and methods that are more easily and economically accessed by more people.

### SUMMARY

In accordance with an aspect, the invention provides a system for capturing diagnostic eye information. The system includes at least one energy source for directing electromagnetic energy into an eye of a subject, a plurality of perception units, each perception unit being associated with an associated position in the visual field of the eye, and each perception unit being adapted to capture refractive information from the eye responsive to the electromagnetic energy, and a processing system for determining refractive error information associated with each position of each perception unit in the visual field of the eye, and for determining refractive error composite information regarding the eye responsive to the refractive error information associated with each perception unit and independent of a direction of gaze of the eye.

In accordance with another aspect, the invention provides a system for capturing diagnostic eye information that includes at least one energy source for directing electromagnetic energy into an eye of a subject, a perception system adapted to capture refractive information from the eye responsive to the electromagnetic energy as well as pupil diameter information representative or a pupil diameter of the eye, and a processing system for determining refractive error information of the eye and associating the refractive error information with the pupil diameter of the eye.

In accordance with a further aspect, the invention provides a system for capturing diagnostic eye information that includes at least one energy source for directing electromagnetic energy into an eye of a subject, a perception system adapted to capture refractive information from the eye responsive to the electromagnetic energy, a partially reflective mirror through which the perception unit is directed toward the eye, an object image that is visible to the subject through the partially reflective mirror, a mirror control system for rotating the partially reflective mirror to change an apparent distance of the object image between a first distance and a second distance, and a processing system for determining refractive error information of the eye and associating the refractive error information with any of the first distance and the second distance.

In accordance with a further aspect, the invention provides an automated eye examination system for capturing diagnostic eye information. The automated eye examination system includes an alignment system for providing alignment information regarding an alignment of a subject with respect to an alignment camera system, a diagnostics analysis system for determining refractive error information associated with at least one eye of the subject within a field of view of the diagnostics analysis system, and an alignment correction system for adjusting the field of view of the diagnostics analysis system responsive to the alignment information.

In accordance with a further aspect, the invention provides a method of capturing diagnostic eye information. The method includes directing electromagnetic energy into an eye of a subject, capturing perception data at each of a plurality of perception units refractive information from the eye responsive to the electromagnetic energy, each perception unit being associated with an associated position in the visual field of the eye, determining refractive error information associated with each position of each perception unit in the visual field of the eye, and determining refractive error composite information regarding the eye responsive to the refractive error information associated with each perception unit and independent of a direction of gaze of the eye.

### BRIEF DESCRIPTION OF THE DRAWINGS

The following description may be further understood with reference to the accompanying drawings in which:

FIG. 1 shows an illustrative diagrammatic view of a configuration of a gaze-independent refractor system for use in a system in accordance with an aspect of the present invention;

FIG. 2 shows an illustrative diagrammatic view of a cluster configuration for gaze-independent photo-refraction in an example of a concentric ring light for pupil diameter steering in accordance with an aspect of the present invention;

FIG. 3 shows an illustrative diagrammatic view of an example of a pattern of probe positions on a retina formed by a cluster configuration where the gaze angle is the offset between the central axis and the gaze direction in accordance with an aspect of the present invention;

FIG. 4 shows an illustrative diagrammatic view of an example of a fitted curve to a spatial map of probe positions, where the value V at the fovea can be determined by finding an extremum (e.g., minimum) of the curve;

FIG. 5 shows an illustrative diagrammatic view of an example of an alternative cluster configuration with eight camera positions providing a configuration with no camera at the center of the cluster that also includes separated pupil steering lights;

FIG. 6 shows an illustrative diagrammatic view of an example of an alternative configuration with a central camera and eccentric IR lights, wherein one ore multiple separate camera(s) and IR lights move along a path at defined intervals during acquisition in accordance with an aspect of the present invention.

FIG. 7 shows an illustrative diagrammatic view of an example of a camera cluster configuration with seven camera positions with an array of IR light sources arranged in a grid in accordance with an aspect of the present invention;

FIG. 8 shows an illustrative diagrammatic view of a vision exam kiosk that houses a system in accordance with an aspect of the present invention;

FIG. 9 shows an illustrative diagrammatic view of an example of components in an input touch display and exam window of a vision exam kiosk in accordance with an aspect of the present invention;

FIG. 10 shows an illustrative diagrammatic view of an example of a pupil diameter steering system with a feedback loop that may be used to control the convergence of the subject's pupil diameter to the target pupil diameter in accordance with an aspect of the present invention;

FIG. 11 shows an illustrative diagrammatic view of an example of a virtual object (target) visible inside the exam window, with the virtual object visually appearing to hover at a set distance inside the exam window interface in accordance with an aspect of the present invention;

FIG. 12 shows an illustrative diagrammatic view of an example of a virtual object system for making a graphical object appear hovering inside the exam window at a far distance from the subject (e.g., 20 feet), wherein the virtual object system may be combined with a photo-refractor in accordance with an aspect of the present invention;

FIG. 13 shows an illustrative diagrammatic view of an example of the virtual object system of FIG. 12, with the partial reflection mirror rotated such that the virtual object now appears at a near distance (e.g. 3 feet) in accordance with an aspect of the present invention;

FIG. 14 shows an illustrative diagrammatic view of an example of a self-administered visual acuity chart that can be implemented in the vision exam kiosk in accordance with an aspect of the present invention;

FIG. 15 shows an illustrative diagrammatic view of alternative variations of the interactive self-administered visual acuity chart in accordance with further aspects of the present invention;

FIG. 16 shows an illustrative diagrammatic view of a subject at an autonomous eye examination kiosk;

FIG. 17 shows an illustrative diagrammatic view of components of the diagnostic system in accordance with an aspect of the present invention; and

FIG. 18 shows an illustrative diagrammatic view of a tracking camera image in the system of FIG. 17.

The drawings are shown for illustrative purposes only.

### DETAILED DESCRIPTION

In accordance with various aspects, the invention provides a new type of refraction technique and eye exam apparatus that remedies the above accuracy limiters while simultaneously enabling automated self-measurement by laypersons in or outside clinical settings. The refraction techniques outlined herein are rooted in the physics of the eccentric photo-refractive principle, a subcategory of the retinal reflex techniques. The refraction techniques may be combined with sensors in a kiosk such that fully automatic refraction can be done after the subject pushes a button to start. Novel visual acuity testing techniques are outlined for inclusion or combination with the novel refraction techniques described herein in a self-serve eye testing kiosk in accordance with an aspect of the present invention.

In illustrative implementations of this invention, the above-mentioned problems with conventional refraction, and in particular photo-refraction, are remediated. In particular, in accordance with an aspect, the invention solves the problem of gaze-misalignment during photo-refraction. In illustrative implementations, a camera-cluster with multiple eccentric light sources performs photo-refraction to measure refractive aberrations, in a manner that is independent of the subject's gaze direction. In some implementations, a photo-refractor is coupled with a pupil diameter control system that steers the pupil diameter at a desired rate or sets the pupil diameter to a desired value. Performing photo-refractive measurement at multiple pupil diameters enables the detection of high order aberrations and setting the pupil diameter to a desired reduced value may limit the influence of high order aberrations. In certain cases, the gaze-independent photo-refraction or the pupil diameter control system is combined with a visual acuity test that is self-administered by the subject. Performing photo-refraction during a visual acuity exam enables improved control over the subject's accommodative state because the subject's focus is engaged and focused at a real or virtual far point (for example 20 ft as per standard Snellen chart distance) or there is more time to acquire multiple photo-refractive measurements that may enable the determination of the accommodative state of the subject's eye or both eyes simultaneously. In further cases, the visual target of the exam apparatus may be set to different distances during refraction or visual acuity testing to gain measurements at eye focus distances which may provide additional information about refractive or accommodative states of the eye.

The overall system may be embedded into a self-serve or ECP-guided vision examination kiosk, or may be compact enough for other tabletop or handheld configurations.

### Gaze-Independent Photo-Refraction

In illustrative implementations, a photo-refractor camera-cluster with multiple cameras and eccentric sources performs photo-refraction to measure refractive aberrations, in a manner that is independent of the subject's gaze direction in accordance with an aspect of the present invention. A camera cluster may be employed to measure the retinal reflex at multiple discrete positions on the retina (probe positions). These probe positions may cover parts of the central and peripheral retinal visual field. The measurement results and relative location of each probed position may be spatially mapped in a scatter plot describing the refractive errors or eye information across the eye's visual field. By fitting a 3-dimensional surface to the map, a continuous curve may be found that models the central and peripheral visual field refractive errors. (This is because, in almost all healthy human eyes, the refractive power changes from central to peripheral vision, and the change is monotonic up to about 25 degrees into the periphery. Due to person-to-person differences in the human eye, the refractive power either increases, remains the same, or decreases monotonically regardless of myopic, emmetropic, and hyperopic eyes.) In illustrative implementations of this invention, each of one or more types of refractive error at central vision are calculated by searching for an extremum of a fitted curve. Which type of extremum (e.g., global minimum, global maximum, local minimum or local maximum) is searched for may depend on, among other things, the type of refractive error and the closeness of the fit for the fitted curve. As discussed herein photo-refraction may be performed independent of the user's gaze direction, in order to measure refractive error of a subject's central and peripheral vision.

A cluster of cameras may be arranged adjacent to each other and each point toward the subject's eyes. The cameras may be positioned along a curved geometric surface (e.g., concave or convex) and may be equidistant from the subject's eyes. Alternatively, the cameras may be positioned on a geometric plane. Each camera may be paired with a combination of eccentric light sources. The light sources may be infrared LEDs (IR). The cluster configuration may form an acceptance cone between the subject's eye and the cameras, and any gaze angle of the subject's eye within the acceptance cone may result in a valid reading. The gaze angle may be the angle between: (a) the eye's visual axis; and (b) the straight line from the eye to the center of the camera cluster (henceforth the “center axis”). The largest gaze angle that results in a reliable photo-refraction reading may occur when the subject's gaze direction is pointing towards the outermost camera of the camera cluster.

Each camera may be surrounded by multiple energy sources (e.g., IR lights). In some cases, the cameras are spaced at equidistant angles from one another. In some cases, the IR lights are oriented along the same geometric surface as the camera cluster surface, or are parallel to or equidistant from that geometric surface. The IR lights may emit IR light that enters and then exits an eye being tested in the eccentric photo-refraction method. The IR lights may be positioned at multiple meridians around the camera, the subject's eyes may be probed for spherical and cylindrical (astigmatic) errors. This may be accomplished by turning each IR light on-off sequentially while recording the double pass retinal reflex present on the subject's pupil. The retinal reflex may be extracted from the IR light pixel intensities of the pupil on the camera images. The refractive state from each IR light on-off cycle may be calculated using one of the common photo-refractor image-to-diopter conversion methods such as the intensity slope-based method or the crescent shape method. Conversion to refractive error from the pupil pixel intensities may also be done via image classifiers from trained neural networks, or by other artificial intelligence (AI) image processing-based classifier techniques. In some cases, the refractive error result is determined by an empirically found lookup table that correlates the value extracted from the pupil pixels via the aforementioned methods to a spherical refractive error (SP: defocus power error in diopters), a cylindrical refractive error (CY: astigmatic power error in diopters), and the angles of cylindrical refractive error (AX: angle of astigmatism in degrees or radians) over a specified range (e.g. −7 to +7 diopters SP or CY).

Each camera and its paired IR lights in the cluster may form an independent photo-refractor that probes a specific position (e.g., very small region) on the retina's visual field. Light from the IR light source may enter the cornea, then pass through the pupil aperture, then the lens, and then translate onto a small region on the retina, then reflect off the retina (retinal reflex) and back through the lens, pupil aperture, and cornea and finally arrive at the camera. In this approach, the IR light passes through the pupil twice, thus we sometimes call it double pass reflex or retinal reflex. With a cluster of cameras and IR light sources, multiple reflex positions on the retina may be probed simultaneously or within a single measurement session. The pattern of positions probed on the retina may form the same pattern as the configuration of the cameras in the cluster. Each probe position on the retina may give a refractive error measurement result in SP, CY, and AX; or higher order aberrations such as trefoil or coma. These results may also be combined into their spherical equivalent value SE in diopters defined as SE=SP+CY/2 (the AX may be discarded).

Having the individual components of the refractive error for each probe position enables the calculation of separate discrete spatial maps each describing the SP, CY, and AX result only. Alternatively, a spatial map of the SE of each probed position may be calculated. The spatial map, or “map”, may be a 3D scatter plot with each point having coordinates (X,Y,V), where X and Y are the position on the retina and V is the value of a given probed point SP, CY, AX, or SE. The unit of X and Y may describe a distance (e.g. mm) or angle on the retina (e.g. degrees or radians). The unit of V may describe the refractive error in diopters, or the absolute power in diopters, or the difference in refractive error to a given calibration constant in the SP, CY, or SE maps, where the greater the refractive value the greater the value V. The unit of V may describe the angle in degrees or radians for the AX map.

Using either the SP, CY, or SE discrete maps, a three-dimensional curve may be fitted to the points on the map. The curve fitting may be performed on SP, CY, or SE discrete maps separately and independently resulting in a SP curve, a CY curve, or a SE curve.

Fitting may be done via a non-linear least squares, linear least squares, least absolute residual, bi-square, polynomial regression, or piece-wise linear regression fitting method. The surface function to fit to the map points may be a predefined polynomial, a nth-order polynomial, 3D spline, or 3D surface from a lookup table forming a continuous spatial map of SP, CY, or SE values. The maps may describe the components of central and peripheral refractive errors of the eye spatially. A curve may be fitted to one of these maps, and an extremum of the fitted curve may provide a refractive error of central vision (at the fovea).

In most eyes, the refractive error of SP, CY, or SE increases or decreases monotonically from the fovea position up to about 25 degrees into the periphery (gaze angle), regardless of myopic, emmetropic, and hyperopic eyes. In other words, an eye's peripheral vision generally has a different refractive power than at central vision. The gaze angle is the angle between the center axis and the eye's visual axis. The center axis is the straight line between the center of the pupil and the center of the photo-refractor cluster. The visual axis is the straight line between the center of the fovea and the center of the pupil. When the eye focuses on the center of the photo-refractor cluster, the center axis aligns with the center of the fovea and the gaze angle is 0. When the eye rotates with respect to the center axis, the center axis may pass through a point on the retina outside the fovea, on the periphery. The gaze angle into the periphery increases in all directions from the fovea with increased eye rotation. A centroid is a non-limiting example of each “center” of a region (e.g., pupil, fovea or camera cluster) that is referred to herein.

For each type of refractive error, a curve describing average central and peripheral refractive errors may be found from empirical datasets and studies for a given population. This curve may look similar to a 3D conical surface or gaussian surface and may be a 3D function with independent variables that enables scaling in the X,Y, and V direction. The empirically derived surface function of central and peripheral vision may be curve fitted to a refractive error map as described above.

In some implementations of this invention: (a) to find the SP refractive error in diopters at the fovea, the value at an extremum of the SP fitted curve is determined; (b) to find the CY refractive error in diopters at the fovea, the value at an extremum of the CY fitted curve is determined; and/or (c) to find the SE refractive error in diopters at the fovea, the value at an extremum of the SE fitted curve is determined. Again, in illustrative implementations, this approach yields highly accurate calculations of refractive error for most humans, because refractive power in most humans increases or decreases monotonically up to about 25 degrees from central to peripheral vision regardless of myopic, emmetropic, or hyperopic central vision refractive error. This approach does not require the subject to adjust their gaze direction towards a central camera to find the refractive error at central vision (the fovea).

In some cases, instead of using a predefined polynomial, a nth-order polynomial may be fitted to the refractive error maps. To find a refractive error (e.g., SP, CY, or SE) at the fovea, the position and value (e.g., SP, CY, or SE) at an extremum of the fitted curve may be determined.

In many use scenarios, one or more computers calculate a particular refractive error of central vision by finding a global maximum or a global minimum of the fitted curve. In some other use scenarios, one or more computers calculate a particular refractive error of central vision by finding a local minimum or local maximum of the fitted curve. As a non-limiting example, in some use scenarios, the computer(s) calculate a very high-resolution polynomial fit, and the point on the fitted curve that corresponds to the fovea is at a local minimum or local maximum of the fitted curve.

In some cases, a 3D interpolation may be performed on maps using the nearest-neighbor, cubic, or linear method or via a Voronoi tessellation forming up-sampled discrete point maps of SP, CY, or SE values. From the discrete point maps, the position and refractive error value (SP, CY, or SE) at the fovea may be found by calculating the center of mass of the map, or by finding one or more extrema on the discrete up-sampled point map. As noted above, depending on the particular use scenario, particular patient and particular refractive error: (a) the extremum that is used to calculate the particular refractive error may be a global minimum, global maximum, local minimum or local maximum; and (b) a computer may determine that a value at the calculated extremum is equal to the particular refractive error.

In some cases, the steps to determine position and value of refractive errors of central and peripheral vision may be repeated over multiple measurement cycles to create a set of retina probed position values, such that multiple curves are created with respect to each set of probed positions. These curves may be averaged spatially to improve the accuracy of the method.

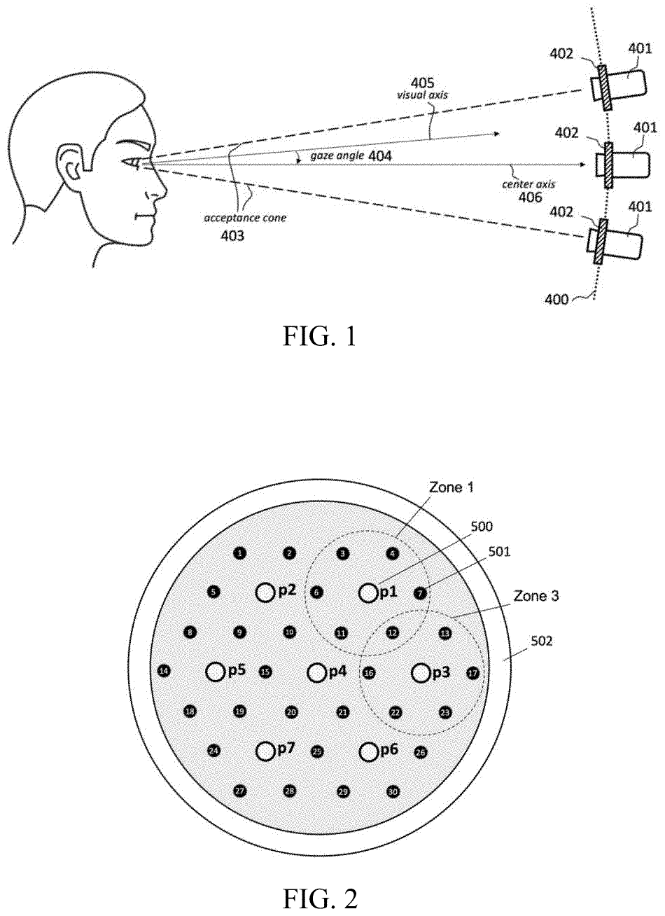

In the illustrative example in FIG. 1, multiple cameras are arranged adjacent to each other on a concave surface 400 and pointing to the subject's eyes. Each camera 401 is paired with a combination of eccentric infrared (IR) light sources 402. The cluster configuration forms an acceptance cone 403 between the subject's eye and the cameras. The gaze angle 404 is the angle between the vector of the eye pointing direction 405 and the center axis 406.

An illustrative example of a camera cluster configuration is shown in FIG. 2. In FIG. 2, multiple eccentric infrared (IR) emitting light sources 501 surround each camera 500. Each of the IR light sources that surround a camera may emit light that travels to the subject's eye (which is being tested) along a path that is at a non-zero acute angle (e.g., greater than 1 degree) relative to the camera's optical axis. Furthermore, depending on the gaze direction of the eye being tested, the optical axis of a particular camera in the cluster may at any given time: (a) be aligned with eye's visual axis or the eye's optical axis; or (b) be at a non-zero angle (e.g., greater than 1 degree) relative to the eye's visual axis or the eye's optical axis or both. Likewise, depending on the gaze direction of the eye being tested, IR light from a particular IR source in the cluster may emit light that travels to the eye along a path that is, at any given time: (a) aligned with eye's visual axis or the eye's optical axis; or (b) at a non-zero angle (e.g., greater than 1 degree) relative to the eye's visual axis or the eye's optical axis or both.

In some cases, there may be cameras arranged in the pattern illustrated in FIG. 2 (position p1 through p7), with each camera surrounded by equally spaced IR light sources 501. For each IR light source on/off cycle, the paired camera may capture the retinal reflex via a camera image and convert the reflex to refractive error results. In FIG. 2, the multiple eccentric IR light sources enable each camera to probe different meridians of the refractive error, thus giving results in Spherical, Cylindrical, and Cylinder Axis components (SP, CY, AX) for the eye being measured.

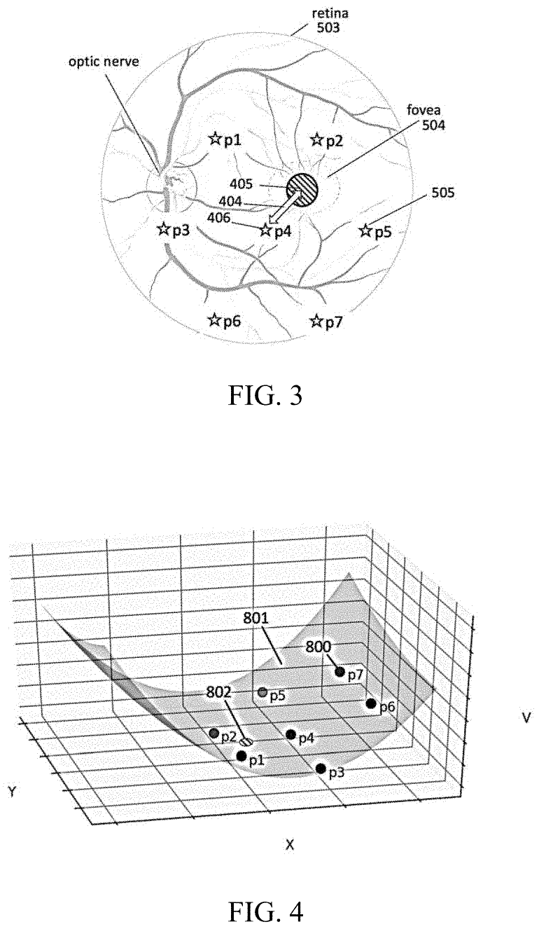

The pattern of camera positions (e.g., FIG. 2, p1-p7) may translate to a pattern of probing positions relating to positions on the retina (e.g., FIG. 3, p1-p7). Each probing position may provide a refractive error measurement (SP, CY, AX). In some cases, the subject's gaze direction 405 may not point to the center of a camera 401 (e.g., FIG. 1), forming a gaze angle 404. In other cases, the subject's gaze direction 405 may not line up with the center axis 406 of the cluster photo-refraction setup forming a gaze angle 404. This gaze angle is translated to the probing positions 505 on the retina 503, as illustrated in FIG. 3. In the illustrative example of FIG. 3, no camera probe position p1-p7 coincides with the foveal area 504 on the retina.

The information (e.g., refractive error values) from each probed position 800 on the retina may be spatially mapped and plotted with coordinates (X,Y,V), where X and Y are the position on the retina, and V is a value of refractive error. The value of V may be spherical power (SP), cylindrical power (CY), or spherical equivalent power (SE) in diopters, or the axis of the cylinder (AX) in degrees or radians. FIG. 4 illustrates an example of how the measurements from the probed positions p1-p7 in FIG. 3 may be spatially mapped and plotted as a scatter plot. In FIG. 4, a polynomial surface 801 is fitted to the probed positions p1-p7. In FIG. 4, the extremum 802 in this curve is a minimum (e.g., global minimum) of the polynomial surface with respect to the axis V. In FIG. 4: (a) the refractive error being measured may be SP, CY or SE refractive error in diopters at central vision (the fovea); and (b) the refractive error may be extracted by locating the position of an extremum (e.g., the global minimum 802) of the fitted curve in the X and Y coordinates, and then recording the V value at that point. (In the preceding sentence, the fitted curve: (a) is an SP curve if the refractive error being measured is SP refractive error; (b) is a CY curve if the refractive error being measured is CY refractive error; and (c) is an SE curve if the refractive error being measured is SE refractive error.) In the example shown in FIG. 4, the located V value (at the extremum 802 of the fitted curve) is the modeled refractive error value of the fovea in diopters. A computer may estimate that the refractive error of the subject's eye is equal to this V value at the extremum.

In some cases, the polynomial surface 801 after fitting to the probed positions p1-p7 may be convex with respect to the axis V, after which the maxima 802 in the curve may be found to determine the refractive error values of central vision.

In some cases, the curved surface 801 that is fit to the probed positions p1-p7 may be a piece-wise linear or piece-wise polynomial curve, or based on a function from a lookup table. In some cases, the surface 801 that is fit to the probed positions p1-p7 may be a plane.

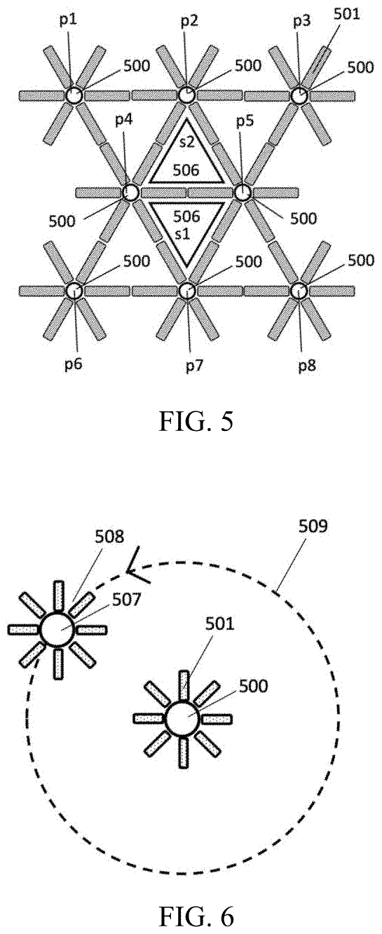

FIG. 5 shows an illustrative alternative example of a camera cluster configuration having camera positions p1-p8. In this example, no camera is positioned at the center of the cluster. In some cases, there may be a stationary camera 500′ and IR light sources 501 pair, combined with a moving camera 507 and IR light sources 508 pair. The moving camera and IR light sources may follow a predefined path 509. The path may be circular, as illustrated in FIG. 6, a spiral path, oval path, rectangular path, or the path may take on any form within the flat plane or surface plane of the cameras 400.

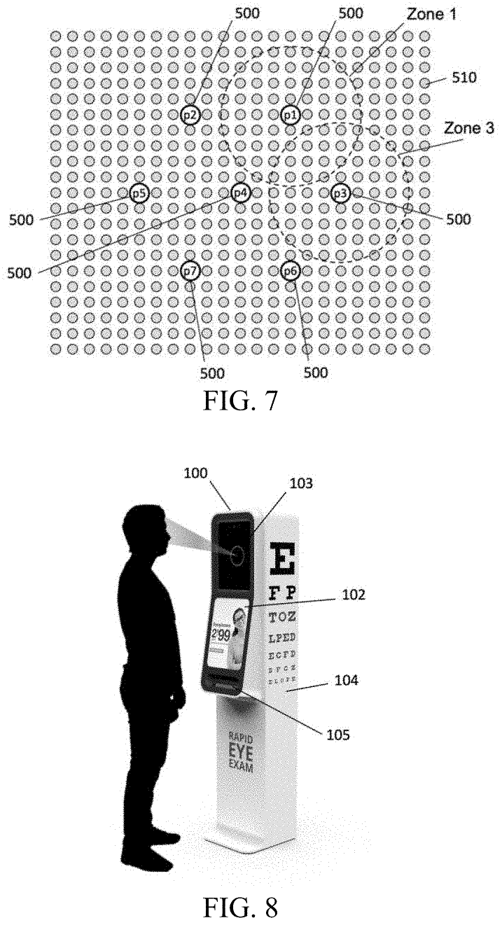

In other cases, the camera cluster and IR light sources may be arranged in a grid as exemplified in FIG. 7. In this example, multiple cameras 500 are arranged in a hexagonal cluster p1-p7, and the IR light sources 510 are arranged in a rectangular grid pattern. The arrangement of the cameras may take on any pattern or arrangement on the cluster surface 400. The number and arrangement of cameras and light sources in the cluster may vary, and is not limited to those shown in FIG. 7. The number and arrangement of cameras may be arranged in a grid or pattern consisting of a multitude of cameras (e.g. 8-by-8, 64 total cameras) with multiple adjacent light sources. The IR light sources may be arranged in a hexagonal grid.

In some implementations of the photo-refractor configurations disclosed in this invention, the light sources may emit near infrared light between 750 nm to 1000 nm wavelengths, or ultraviolet light between 250 nm to 450 nm, or a broadband white light across the visible spectrum, or combinations of specific wavelengths in the visible spectrum.

### Exam Kiosk

In some cases, a self-serve vision exam kiosk 100 houses some of the apparatus and may be employed as a platform to deliver vision tests. FIG. 8 illustrates an example of a kiosk. In FIG. 8, the kiosk 100 contains a touch display console 102 for the subject to input information and for control to the system, an exam window 103, and a housing 104 for the vision exam apparatus. The exam window 103 may face the user's eyes and may interface with a self-serve visual acuity test system (e.g. FIG. 14), or a photo-refractor system (e.g. FIG. 12), or a pupil diameter steering system (e.g. FIG. 10). The kiosk 100 may also contain a lensmeter system 105 for the measurement of eyeglass lens settings.

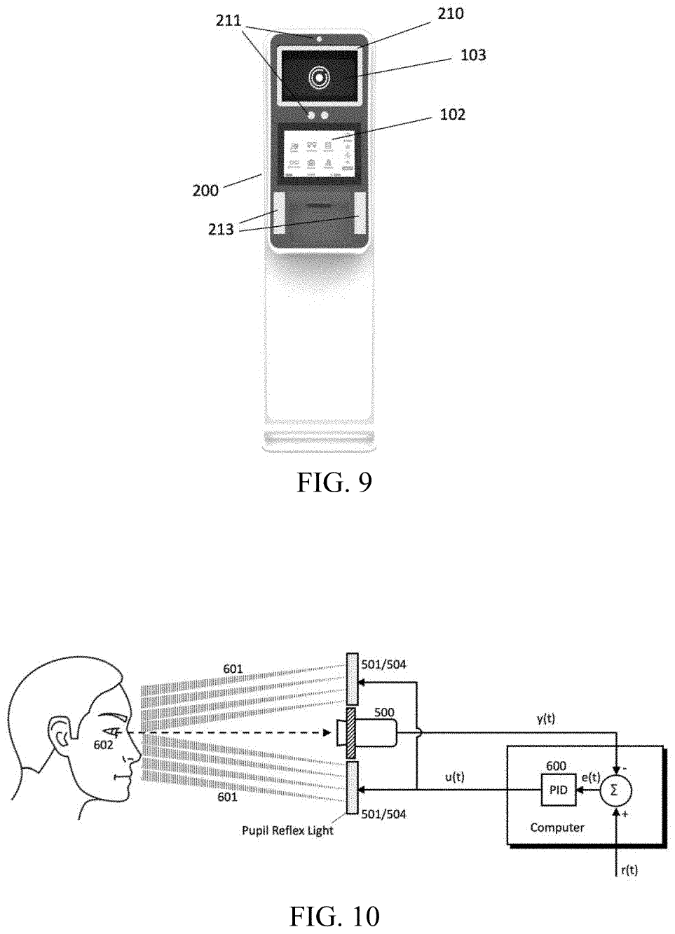

In FIG. 9, the kiosk 200 includes input touch display 102 and exam window 103, both facing the user during testing. The touch display 102 may be used for inputting subject demographic data and medical history, exam selection menu, payment data input, subject contact information input, scheduling of follow-up exam with vision care professional, results display, and test procedure command input. As a general matter, the exam window 103 is where the subject sees letters and symbols at a virtual distance away from subject's eyes (typically 20 ft or similar). The exam window 103 may also interface with the embodiments of the gaze-independent photo-refraction systems described in this invention. The photo-refractor system may also be integrated with the visual acuity system where both systems interface with the same exam window 103. A light source 210 positioned at the border to the exam window 103 or placed inside the kiosk behind the exam window 103 provides visible wavelength stimulus light to the subject's eyes to decrease the pupil diameter of the subject in a controlled manner by selectively adjusting the brightness (e.g. via pupil diameter steering system described below). Multiple cameras or sensors may be embedded into the exam console 211 or behind the exam window 103 to track the subject's head position, eye movement, or pupil diameters with respect to the kiosk. The embedded cameras or sensors 308 may track body posture, head tilt, hand gestures, or presence of specific items such as eyeglasses, contact lenses, or obstructions to the face and eyes. The detection of above examples may be accomplished with artificial intelligence (AI) computer vision classifiers via one or multiple kiosk-embedded cameras. Multiple cameras 308 may be employed simultaneously to detect distance of specific objects on the subject, or features of the subject, via stereoscopic computer vision techniques. Measuring distance from kiosk to subject may also be accomplished via dedicated time of flight (TOF) sensors, ultrasound sensors, or pattern projection techniques via the positional sensor(s) 308. The detected features, objects, or distances of or on the subject may be used as input to the refraction technique, pupil diameter steering, or the visual acuity testing process.

In typical implementations, the apparatus uses the various cameras and sensors of the kiosk combined with the refraction technique to automate the refraction measurements or visual acuity test. The subject or the ECP simply presses a button on the display 102 to start the exam and the system takes over to complete the measurements automatically. During the exam the subject may be asked to stand still while the kiosk adapts to the subject's position while the apparatus takes refraction readings automatically, or the subject may be asked to follow prompts as in the case of a visual acuity test.

An audio feedback speaker bar 213 enables the kiosk to provide virtual assistant audio feedback. The virtual assistant audio tracks welcome and instruct the user to perform certain tasks during the tests performed at the kiosk. The kiosk is therefore fully automated, including camera detection systems that capture both refractive information as well as visual acuity information. The system may be trigger by either a single start button or simply the presence of a subject standing in front of the system (such as a kiosk). Once initiated the system will automatically align with the subject, and perform diagnostic and visual acuity analyses (potentially even simultaneously), as well as detection of any of a variety of health issues regarding a subject based on, for example, a subject's pupils reactions to changes in visible light.

### Pupil Diameter Steering

As noted above, in some implementations, this invention controls the diameter of the pupil of an eye being tested. The system may comprise an adjustable brightness control light facing the subject, a camera that faces the subject to record the pupil diameter, and a control system that steers the current pupil diameter to the target pupil diameter. The control system may be running on a computer or on a microcontroller. In a healthy eye and subject, a high intensity light entering the eye causes the pupil to constrict via a process called myosis, whereas a low intensity light entering the eye or no light at all causes the pupil to dilate via a process called mydriasis.

In illustrative implementations of this invention, the pupil diameter may be reduced by increasing the intensity of the control light. The pupil diameter may be increased by lowering the intensity of the control light. The ambient light may be reduced by turning off the room lights, or by shielding the eye from ambient light via a booth or kiosk design that includes side blinders, or a chamber design with an enclosure to block light to the subject. The wavelength of the control light is in the visible range of the human eye so myosis may occur and may be characterized as a chromatic color such as a red, green, or blue, or as an achromatic color such as white or gray. The control light may be diffusely propagating and may enter both eyes simultaneously, or the control light may be focused to shine light into one eye at a time.

The pupil diameter steering method may include a control system that sets the intensity of the control light so that the pupil reaches the target pupil diameter. The control system may include an open loop controller that takes in the target pupil diameter and sets the intensity of the control light from a lookup table that relates light intensity to pupil diameters. The lookup table may take into account age, gender, race, wavelength, light source spatial configuration, and if light is being delivered to one eye or both eyes simultaneously. The open loop controller may wait an estimated amount of time until the desired range of pupil diameter is reached.

In some cases, the control system includes a closed loop feedback controller that takes in a target pupil diameter value and compares to the current pupil diameter value measured by the camera to then actively drive the intensity of the control light. The closed loop feedback controller may be a proportional controller, a proportional-integral-derivative controller (PID), a state-space feedback controller, or a fuzzy logic controller. The feedback controller may also be a multi-loop closed-loop feedback controller. In certain applications, the control system takes in a target pupil diameter rate-of-change value and drives the control system to change the subject's pupil diameter at the target rate of change.

FIG. 10 illustrates an example of a PID closed-loop feedback controller that drives the pupil diameter steering method. The target pupil diameter value r(t) is entered to the control loop. Each camera 500 records images of the subject's eye 602 and the current pupil diameter y(t) is extracted via image processing techniques. A PID closed-loop feedback controller 600 takes the target diameter r(t) and subtracts the current diameter y(t) to give e(t). The controller dampens or amplifies e(t) to give u(t), the signal that drives the intensity of the control light 501/504. In certain cases, the pupil diameter steering system includes multiple cameras that face the subject's eyes, so that the eye and pupil diameter may be recorded from multiple vantage points. An example with multiple cameras is illustrated in FIGS. 2, 5, 6, and 7, where the photo-refractor cameras double as pupil diameter recording devices that provide y(t) to the feedback controller.

In further applications, the control light comprises multiple diffuse area light sources spaced adjacent from one another as illustrated in FIG. 5 (s1, s2), or a cluster of point-like sources arranged in a grid as illustrated in FIG. 7 (510), or one or multiple laser light sources pointing to the subject's eye or both eyes. The control light may be a ring light as illustrated in FIG. 2 and may comprise a series of LEDs that are arranged in a circle and covered by a diffuser plastic or glass material to smoothen the uniformity of the emitted light. The control light may take the form of triangular or circular area LED lights as illustrated in FIG. 5 (506). Significant monochromatic high order aberrations in the eye may cause unwanted shifting of the retinal reflex-to-diopter conversion tables used in photo-refraction and therefore may significantly affect the accuracy of the measurement.

In accordance with certain aspects of the present invention (e.g., with a gaze-independent photo-refractor), pupil diameter steering is employed to detect high order aberrations. In some implementations, higher order aberrations are detected as follows: A computer: (a) may determine whether the cylinder axis angles map shows that the AX values of the retina probe positions are pointing in different directions; and (b) if they are, may determine that there are significant coma or trefoil aberrations. This approach yields accurate results because, in a typical eye with spherical (SP) and cylindrical (CY) refractive error (low order aberrations) but without significant high order aberrations, the cylinder axis angles (AX) of central and peripheral vision tend to point in the same direction.

In some implementations, higher order aberrations are detected by deliberately reducing the pupil diameter of the subject's eye during measurement (e.g., in a series of different steps of diameter of the pupil). Reducing the pupil diameter tends to cause the photo-refractive measurement to be less affected by high order aberrations in the eye. This effect may be used to compare the refractive error between a dilated pupil and a constricted pupil via the pupil diameter steering system. A significant difference in refractive error between a constricted pupil and a dilated pupil may indicate the presence of high order aberrations.

In some implementations, the magnitude of high order aberrations are determined by taking the cylinder refractive error CY from the center of the map or the fitted curve and comparing it to the periphery CY values. The difference in diopters between center and periphery CY values may be correlated to a table of magnitudes of trefoil and coma residual aberrations.

In accordance with certain aspects of this invention (e.g., with a gaze-independent photo-refractor), pupil diameter steering is employed to detect symptoms of eye disease or neurological disorders such as asymmetric pupil diameters (i.e. anisocoria) between both eyes, non-responsive pupils, abnormal rate of change of pupil diameter, or pulsating pupil diameters.

### Virtual Object

In some implementations, the user may see a graphic (“virtual object” 306) appearing inside the exam window 300. The virtual object may be a fixed graphic, an animated graphic, or a combination of both. The virtual object may be used to guide the subject's attention, focus position, or eye gaze direction to a specific location when looking into the exam window. To the subject, the virtual object may appear at a given distance behind the exam window 300 as if “hovering” inside. In some implementations of the kiosk, the virtual object may be combined with a photo-refractor, or a gaze-independent-photo-refractor configuration disclosed herein, or a visual acuity system to serve as a viewing target to assist in administering the tests.

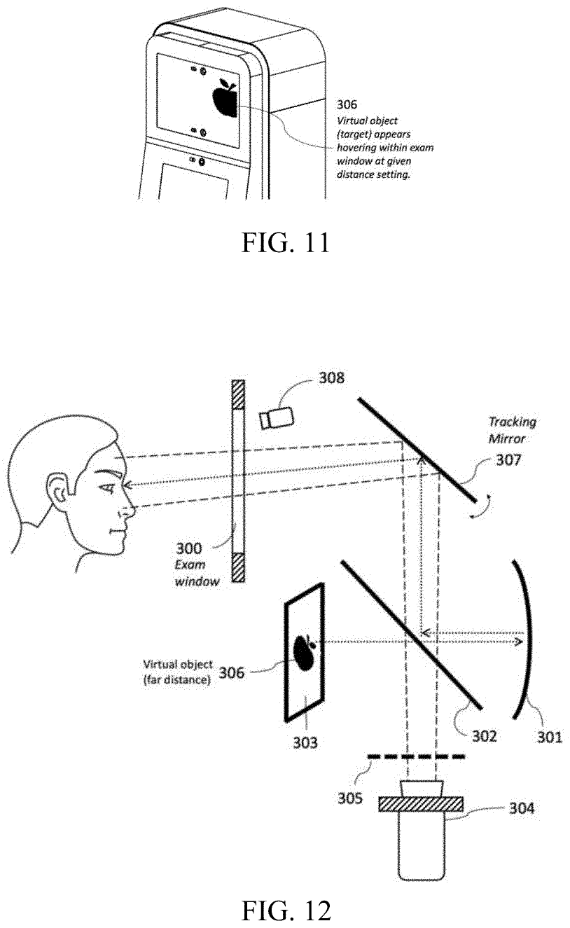

FIG. 12 illustrates an implementation of an optical system to create the virtual object. The system may be composed of an exam window 300, a parabolic mirror 301, a partial reflection mirror 302, and a virtual object display 303. In this embodiment, the virtual object 306 can be made to appear at a far distance from the subject (e.g. 20 ft) in a kiosk that has exterior dimensions shorter than the virtual object distance as shown in FIG. 11. This may be accomplished by the optical path compression configuration illustrated in FIG. 12. The optical path of the system originates at the virtual object display 303 and is folded and refocused until meeting the subject's eyes. In more detail, light rays travel from the virtual object display 303 first through a partially reflective mirror 302 angled 45-degrees to the optical axis of the system, then encounter a concave parabolic mirror 301 that focuses the rays, then redirect from the partially reflective mirror 302, then pass through the exam window 300 and finally reach the subject's eyes. This configuration creates a “hover” effect where the virtual object appears straight ahead when looking into the exam window, even though the source of the image—the virtual object display—is not located at the same visual position. A benefit of this configuration is to enable a volumetrically compressed kiosk form factor while retaining the ability to display virtual objects at a far viewing distance.

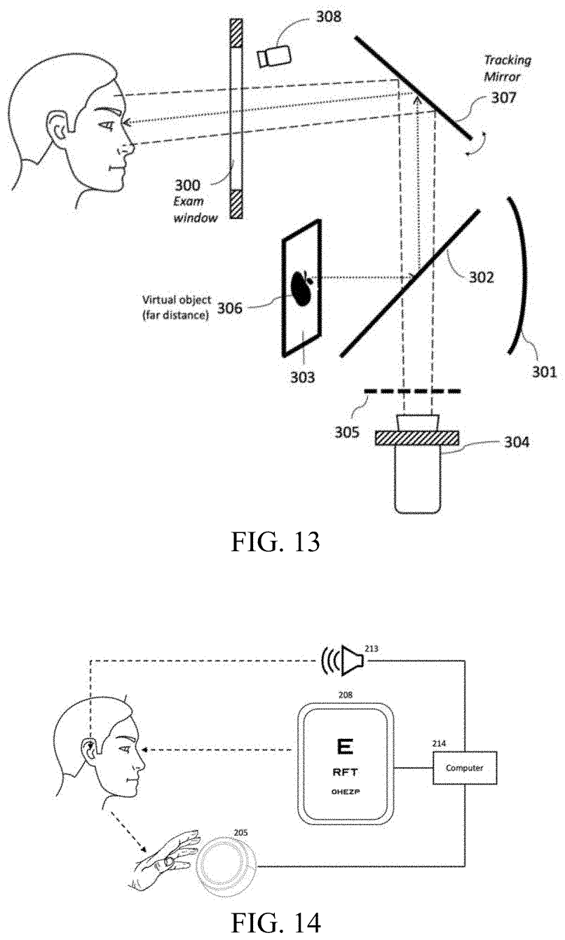

In some implementations, the partial reflection mirror can be rotated to such that the virtual object appears at an alternative distance. FIG. 13 illustrates an example where the virtual object appears approximately 3 feet away from the subject. In more detail, light rays travel from the virtual object display 303 to the partially reflective mirror 302 and redirect through the exam window 300 and then reach the subject's eyes. This configuration creates a “hover” effect where the virtual object appears straight ahead when looking into the exam window, even though the source of the image—the virtual object display—is not located at the same visual position.

The partial reflection mirror 302 may be a glass or plastic slab and covered on one or both sides with specialty materials, films, or optical coatings. The slab may be covered with an optical filter coating that passes and reflects certain wavelengths of light, or an anti-reflection coating, or an optical absorber coating, or a polarizer film or coating. The partial reflection mirror may be a beam-splitter mirror a or a teleprompter mirror with anti-reflective coating on one or both sides. In some implementations, the slab reflection and transmission ratio for a given wavelength is 50% reflection and 50% transmission, or 40% reflection and 60% transmission, but can be any ratio combination between reflection and transmission.

The optical path compression configuration exemplified in FIG. 12 or in FIG. 13 may be used for a self-administered visual acuity exam system (described in more detail below), or combined with a common photo-refractor 304 or with a photo-refractor embodiment described in this invention 304 for simultaneous visual acuity testing and eye refraction, or for displaying a virtual object at a position of interest for refraction by a photo-refractor 304. The photo-refractor may be combined with an optical filter 305 to remove unwanted wavelengths of light from entering the photo-refractor sensor. In some implementations, the distance to the virtual object seen by the subject may be the same as the optical distance from subject to photo-refractor. This can be accomplished by rotating the partial reflection mirror 302 to the position illustrated in FIG. 13 and setting the distance of light rays between partial reflection mirror 302 to the virtual object display 303 to the same distance as between the partial reflection mirror 302 and the photo-refractor sensor 304.

The optical systems illustrated in FIGS. 12 and 13 may be combined simply by actuating the partial reflection mirror 302 such that it can be rotated during or before the test to create two separate virtual object distances. This allows the photo-refractor to refract the subject's eyes at two separate accommodative states (focus distances). In one implementation, the virtual object may be at a 3 foot distance, and then switched to a 20 foot distance by simply rotating the partial reflection mirror by 90 degrees. The photo-refractor may take multiple measurements at both virtual object distances.

The position of the virtual object display 303, or the parabolic mirror 301 may also be moved relative to the optical axes or other components to create additional virtual object distances and accommodative states of the subject's eyes.

The virtual object size on the display 303 may be coupled to the value received from the positional sensor 308. This enables the adjustment of the size of the virtual object 306 regardless of the position of the subject with respect to the kiosk. That is, the virtual object 306 can be made to appear the same size regardless of the subject's standing position (e.g. for subjects standing close to the exam window 300 the virtual object 306 is reduced in size, and for subject standing further away to the exam window 300 the virtual object is increased in size).

The angle of the partial reflection mirror 302 may also be coupled to the value received from the positional sensor 308 such that the virtual object 303 always appears in a specific position inside the exam window 300 regardless of the subject's head height or position with regards to the kiosk. For example, for a subject that is short the partial reflection mirror 302 can rotate such that the virtual object 306 moves up, and for a subject that is tall the partial reflection mirror 302 can rotate such that the virtual object 306 moves down.

In some implementations, the above dynamic virtual object positioning methods may be employed during a visual acuity test, or during auto-refraction with a photo-refractor, or with a gaze-independent-photo-refractor. The size and position of the virtual object as it appears inside the exam window is therefore adjustable depending on where the subject is standing or is otherwise positioned. This ensures that all users see the same object (regardless of their height or position near the exam window.

### Monitoring and Controlling Accommodation

In some implementations, the combination of a virtual object and a photo-refractor may be used to monitor a subject's accommodative state. As the subject performs the test routine, the photo-refractor may continuously measure the refractive state of the subject's eyes and record the results over time. The time series may show relative changes in refractive power as the subject is changing their accommodative state. The visual acuity test may prompt the subject to attempt their best focusing ability on the virtual object seen inside the exam window. Tracking a refractive error time series may be used to estimate the subject's best focusing ability by identifying the maximum or minimum value on the time series graph.

Multiple accommodative states may be monitored during a measurement session by taking photo-refractor measurements at a multitude of virtual object distances and utilizing the aforementioned virtual object positioning methods. For example, the virtual object distance is first set to 3 feet from the subject and then refractive measurements are taken. Next, the virtual object distance is set to 20 ft and then refractive measurements are taken. If both refractive measurements are the same or similar, it may indicate a high probability that the user has focused at the correct distances and the results are valid. If the refractive measurements are not the same between two virtual object distances, that may indicate issues with accommodating by the subject.

Correctly accommodating to a target distance of a virtual object may also be an issue depending on what type of refractive error the subject has. For farsighted subjects (hyperopic) a virtual object 3 feet away may appear too blurry to correctly focus at that distance. The virtual object may instead be set to a further distance, such as the typical Snellen chart distance of 16 ft or 20 ft, so that the subject can more reliably focus on the virtual object and therefore the photo-refractor can get a measurement with a higher probability of being valid. For nearsighted subjects (myopic), the opposite strategy may be employed. A 20 ft virtual object distance may be too blurry to properly focus, so instead the virtual object distance may be set to 3 feet for easier focus and improved measurement reliability.

### Self-Administered Visual Acuity Test

A self-administered visual acuity test may be performed by having the subject (kiosk user) follow instructions from the kiosk and input responses back to the kiosk as illustrated in FIG. 14. From the responses the subject's visual acuities may be determined. The system may include a dynamic eye chart 208 created by the virtual object 306 that the subject sees through the exam window 103 at a simulated set distance, such as 20 ft. The letters or symbols of the virtual object 306 appear to the subject to be at a far distance, when in reality the system is made compact via an optical path compression method displaying objects at a virtual distance (such as shown, for example, in FIG. 12). The visual acuity test may be performed without the assistance of an ECP, or with guidance from an ECP standing next to the kiosk, or virtually by an ECP communicating to the subject via a kiosk teleconferencing system.

During the test, the subject may be prompted by the kiosk to perform an action or a series of actions that the kiosk receives as input. This input may then be used to adjust the virtual object 306 in size, shape, or position as seen in the exam window 103.

In one implementation, the subject may be prompted via audio commands coming from the kiosk console's loudspeaker 213 to rotate an input wheel 205 after observing the virtual object 306. Turning the input wheel or pushing the wheel's button adjusts the letter(s), symbol(s), or graphic(s) of the virtual object 306 to new letter(s), symbol(s), or graphic(s), and/or to a new size, position, or initiates a prompt to a new step in the test.

Instead of providing audio commands to the subject to advance to the next step, the kiosk may prompt the subject to perform a new test action via graphics or text on the kiosk's display or via letters, symbols (e.g. arrows), or graphics displayed by the virtual object 306.

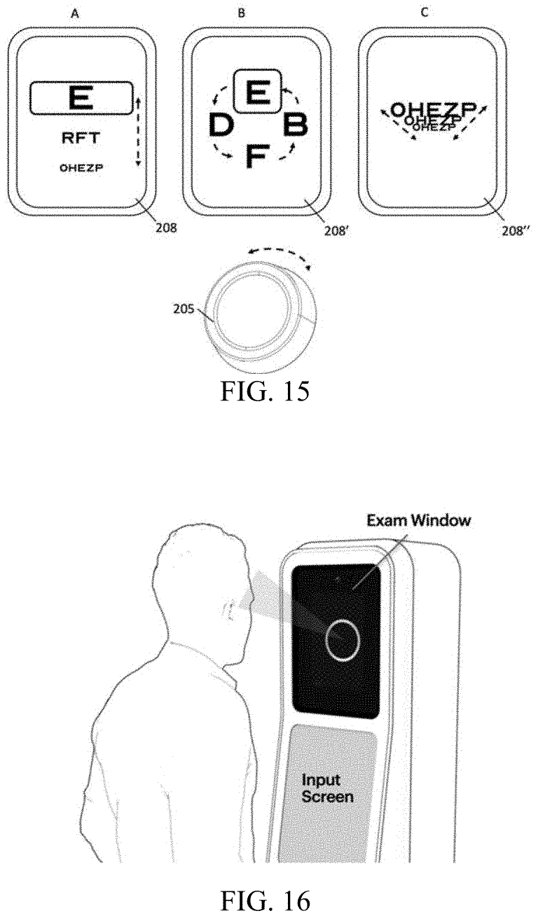

The central computer 214 housed inside the kiosk 200 reacts to the subject's input by advancing the test step and outputs audio commands or a change in the virtual display's state. FIG. 15 exemplifies different implementations of the dynamic visual acuity test using the configuration outlined in FIG. 14.

In one implementation, instead of using the input wheel 205, the subject may input a response to the test by pressing buttons on the kiosk's touch display, or by performing a “swiping” action on the touch display, or by pressing a touch pad with buttons on the kiosk, or by performing gestures that the cameras or sensors 308 can detect (e.g. head nodding, head turning, hand waving, hand positions, holding up all or some fingers, eye blinking, mouth opening or closing).

In accordance with further aspects, the system may detect any of a wide variety of gestures that indicate (positively or negatively) whether a subject is able to clearly see information in a visual acuity test. Such gestures may include, for example, nodding or shaking their head, or providing a thumbs-up or side-to-side movement of a horizontal hand with the palm facing downward. In accordance with further aspects, the system may detect voice of a subject answering yes/no questions and/or reading text in lines during a visual acuity test.

In the examples of FIG. 15, the subject has their vision fixated into the window 208 and is prompted via audio 213 or the touch display 103 to look at the list of letters displayed. The letters are displayed at decreasing size for each line, and the subject is prompted via audio 213 or the touch display 103 to select the line that the subject finds barely readable using the input wheel 205 or buttons on the touch display 103. Selections of a position are made by the subject pressing the input wheel's button 205 or buttons on the touch display 103. The selected line of lettering or symbols by the subject may be used to determine the visual acuity of the eyes. To test one eye at a time, the subject is prompted to hold one eye closed with their hand, while using the other hand to control the input wheel 205 or buttons on the touch display 103. To test the other eye, the hand positions are reversed, such that the opposite hand covers the opposite eye and the free hand holds the input wheel 205 or pushes buttons on the touch display 103. Alternatively the lettering or symbols may all be the same size for each line where the audio 213 or the touch display 103 prompts the subject to select the line that matches a series of letters that are played back via the audio 213. If the subject selects the correct line, a new list of letters or symbols is displayed and the subject is prompted again to select the line with a specific combination of letters or symbols. This may be repeated until the subject cannot reliably select the correct line—a test cutoff point is determined where the subject's rate of correct selections determines the size of objects at a distance that the subject's eye may resolve (visual acuity). The final determined size of letters or symbols are then translated into visual acuity numbers for the tested eye.

In some cases, the rotation of the input wheel 205 or buttons on the touch display 103 enables the subject to move letters or symbols shown in window 208 along a circular path, or rotate the orientation of letters or symbols, where for example, the top positioned letter or symbol is the selection made by the subject as illustrated at 208′ in FIG. 15. As an alternative, the subject may rotate the selection box to the target object letter or symbol prompted by the audio command 213. Selections of a position are made by the subject pressing the input wheel's button 205 or buttons on the touch display 103. The subject may repeat these steps while the letters or symbols reduce in size at each iteration until the visual acuity numbers for the tested eye may be determined.

The subject for example, may rotate the input wheel 205 or press buttons on the touch display 103 such that the letters or symbols seen through the window 208 adjust in size in response to the input wheel rotation direction as illustrated at 208″ in FIG. 15. The subject may rotate the input wheel or press buttons on the touch display 103 until the letters are barely readable and then press the button of the input wheel 205 or buttons on the touch display 103 to select the setting. From the selected rotation position, the visual acuity of the tested eye may be determined.

In some cases, the cameras or sensors 308 may detect if the subject is holding their hand in front of their eye at the appropriate point in the test. If the hand is not in the right position, the kiosk may prompt the subject move the hand back in front of the eye. Further, the cameras or sensors 308 may detect that the subject is not wearing eyeglasses or contact lenses when they are required for the test, and may be prompted by the kiosk to wear the eyeglasses or contact lenses. In some cases, the cameras or sensors 308 may detect that the subject is wearing eyeglasses or contact lenses when they are not supposed to for the test and are then prompted by the kiosk to remove the eyeglasses or contact lenses.

The self-administered visual acuity test may also be combined with the auto-refraction techniques described above. In one example, the subject may follow the visual acuity test while the refraction sensor is recording refractive data continuously over the periods of seconds or minutes. In accordance with further aspects, the system may determine whether a subject is wearing glasses, and confirm whether a subject is blocking one or the other eye when prompted to do so during an automated visual acuity test.

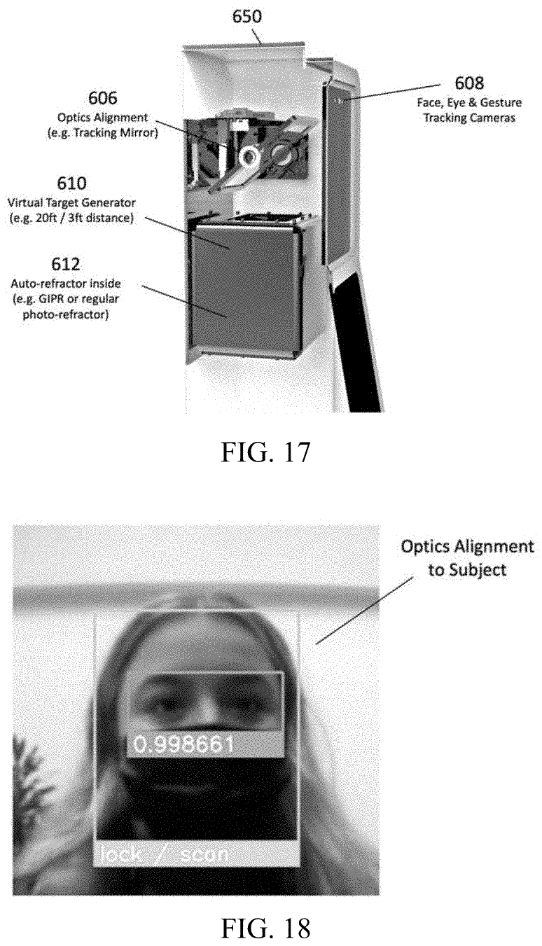

FIG. 16 shows a subject in front of an autonomous examination kiosk 650 in accordance with an aspect of the present invention that includes an exam window 602 through which the subject may see target images, have eye diagnostics performed, and undergo a visual acuity test using, for example, a input screen 604. FIG. 17 shows the inside components of the kiosk 650 including the optics alignment system including the tracking mirror, the face, eye & gesture cameras 608, the visual target generator 610 and the auto-refractor sensor (e.g., GIPR, or regular photo-refractor). With reference to FIG. 18, the system will track and locate the subject's face using artificial intelligence tracking software as discussed above, and the system will then locate the subjects eyes using the GPIR sensors as also discussed above.

### Definitions

The terms “a” and “an”, when modifying a noun, do not imply that only one of the noun exists. For example, a statement that “an apple is hanging from a branch”: (i) does not imply that only one apple is hanging from the branch; (ii) is true if one apple is hanging from the branch; and (iii) is true if multiple apples are hanging from the branch.

To say that a calculation is “according to” a first equation means that the calculation includes (a) solving the first equation; or (b) solving a second equation, where the second equation is derived from the first equation. Non-limiting examples of “solving” an equation include solving the equation in closed form or by numerical approximation or by optimization.

To compute “based on” specified data means to perform a computation that takes the specified data as an input.

Non-limiting examples of a “camera” include: (a) a digital camera; (b) a digital grayscale camera; (c) a digital color camera; (d) a video camera; (e) a light sensor, imaging sensor, or photodetector; (f) a set or array of light sensors, imaging sensors or photodetectors; (h) a light field camera or plenoptic camera; (i) a time-of-flight camera; and (j) a depth camera. In some cases, a camera includes any computers or circuits that process data captured by the camera.

The term “comprise” (and grammatical variations thereof) shall be construed as if followed by “without limitation”. If A comprises B, then A includes B and may include other things.

Each of the following is a non-limiting example of a “computer”, as that term is used herein: (a) a digital computer; (b) an analog computer; (c) a computer that performs both analog and digital computations; (d) a microcontroller; (e) a microprocessor; (f) a controller; (g) a tablet computer; (h) a notebook computer; (i) a laptop computer, (j) a personal computer; (k) a mainframe computer; and (l) a quantum computer. However, a human is not a “computer”, as that term is used herein.

“Defined Term” means a term or phrase that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

“For instance” means for example.

To say a “given” X is simply a way of identifying the X, such that the X may be referred to later with specificity. To say a “given” X does not create any implication regarding X. For example, to say a “given” X does not create any implication that X is a gift, assumption, or known fact.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

Unless the context clearly indicates otherwise, “or” means and/or. For example, A or B is true if A is true, or B is true, or both A and B are true. Also, for example, a calculation of A or B means a calculation of A, or a calculation of B, or a calculation of A and B.

The term “such as” means for example.

Except to the extent that the context clearly requires otherwise, if steps in a method are described herein, then the method includes variations in which: (1) steps in the method occur in any order or sequence, including any order or sequence different than that described herein; (2) any step or steps in the method occur more than once; (3) any two steps occur the same number of times or a different number of times during the method; (4) one or more steps in the method are done in parallel or serially; (5) any step in the method is performed iteratively; (6) a given step in the method is applied to the same thing each time that the given step occurs or is applied to a different thing each time that the given step occurs; (7) one or more steps occur simultaneously; or (8) the method includes other steps, in addition to the steps described herein.

Headings are included herein merely to facilitate a reader's navigation of this document. A heading for a section does not affect the meaning or scope of that section.

This Definitions section shall, in all cases, control over and override any other definition of the Defined Terms. The Applicant or Applicants are acting as his, her, its or their own lexicographer with respect to the Defined Terms. For example, the definitions of Defined Terms set forth in this Definitions section override common usage and any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. Unless the context clearly indicates otherwise, any definition or clarification herein of a term or phrase applies to any grammatical variation of the term or phrase, taking into account the difference in grammatical form. For example, the grammatical variations include noun, verb, participle, adjective, and possessive forms, and different declensions, and different tenses.

### VARIATIONS

This invention may be implemented in many different ways.

Each description herein of any method, apparatus or system of this invention describes a non-limiting example of this invention. This invention is not limited to those examples, and may be implemented in other ways.

Each description herein of any prototype of this invention describes a non-limiting example of this invention. This invention is not limited to those examples, and may be implemented in other ways.

Each description herein of any implementation, embodiment or case of this invention (or any use scenario for this invention) describes a non-limiting example of this invention. This invention is not limited to those examples, and may be implemented in other ways.

Each Figure, diagram, schematic or drawing herein (or in the Provisional) that illustrates any feature of this invention shows a non-limiting example of this invention. This invention is not limited to those examples, and may be implemented in other ways.

The above description (including without limitation any attached drawings and figures) describes illustrative implementations of the invention. However, the invention may be implemented in other ways. The methods and apparatus which are described herein are merely illustrative applications of the principles of the invention. Other arrangements, methods, modifications, and substitutions by one of ordinary skill in the art are also within the scope of the present invention. Numerous modifications may be made by those skilled in the art without departing from the scope of the invention. Also, this invention includes without limitation each combination and permutation of one or more of the items (including any hardware, hardware components, methods, processes, steps, software, algorithms, features, and technology) that are described herein.

What is claimed is:

1. A system for capturing diagnostic eye information, said system comprising:
at least one energy source for directing electromagnetic energy into an eye of a subject;
a plurality of perception units, each perception unit being associated with an associated position in the visual field of the eye, and each perception unit being adapted to capture refractive information from the eye responsive to the electromagnetic energy; and
a processing system for determining refractive error information associated with each position of each perception unit in the visual field of the eye, and for determining refractive error composite information regarding the eye responsive to the refractive error information associated with each perception unit and independent of a direction of gaze of the eye.

2. The system as claimed in claim 1, wherein at least one energy source is provided among a plurality of energy sources that are positioned at a plurality of locations in the visual field of the eye.

3. The system as claimed in claim 2, wherein the plurality of energy sources are spaced from one another, and each of which is associated with at least one perception unit.

4. The system as claimed in claim 1, wherein each energy source is individually engageable.

5. The system as claimed in claim 1, wherein the refractive error composite information includes any of spherical error (defocus) information, cylindrical error (astigmatism) information and cylindrical axis information, and high order aberration errors including any of trefoil error and coma error.

6. The system as claimed in claim 5, wherein the refractive error composite information regarding the eye generally includes spatial mapping information responsive to the refractive error of the inner and peripheral visual field.

7. The system as claimed in claim 6, wherein the spatial mapping is performed via any of non-linear least squares, linear least squares, least absolute residual, bi-square, polynomial regression, or piece-wise linear regression fitting methods.

8. The system as claimed in claim 6, wherein a surface function to fit to map points of the spatial mapping may be a predefined polynomial, a nth-order polynomial, 3D spline, or 3D surface from a lookup table forming a continuous spatial map of spherical error, cylindrical error, cylindrical axis, coma error, trefoil error, or spherical equivalent error information.

9. The system as claimed in claim 1, wherein the refractive error composite information describes components of inner and peripheral refractive errors of the eye's visual field.

10. A system for capturing diagnostic eye information, said system comprising:
at least one energy source for directing electromagnetic energy into an eye of a subject;
a perception system adapted to capture refractive information from the eye responsive to the electromagnetic energy as well as pupil diameter information representative or a pupil diameter of the eye; and
a processing system for determining refractive error information of the eye and associating the refractive error information with the pupil diameter of the eye.

11. The system as claimed in claim 10, wherein the processing system further includes a control system in communication with the at least one energy source and the pupil camera for causing the eye of the subject to achieve a target pupil diameter.

12. A system for capturing diagnostic eye information, said system comprising:
at least one energy source for directing electromagnetic energy into an eye of a subject;
a perception system adapted to capture refractive information from the eye responsive to the electromagnetic energy;
a partially reflective mirror through which the perception unit is directed toward the eye;
an object image that is visible to the subject through the partially reflective mirror;
a mirror control system for rotating the partially reflective mirror to change an apparent distance of the object image between a first distance and a second distance; and
a processing system for determining refractive error information of the eye and associating the refractive error information with any of the first distance and the second distance.

13. The system as claimed in claim 12, wherein the system includes a partially reflective mirror through which at least one perception unit is directed toward the eye, an object image that is visible to the subject through the partially reflective mirror, and a mirror control system for rotating the partially reflective mirror to change an apparent distance of the object image.

14. The system as claimed in claim 13, wherein the system further includes a parabolic mirror off of which the object image may selectively be reflected by rotating the partially reflective mirror.

15. An automated eye examination system for capturing diagnostic eye information, said automated eye examination system comprising:
an alignment system for providing alignment information regarding an alignment of a subject with respect to an alignment camera system;
a diagnostics analysis system for determining refractive error information associated with at least one eye of the subject within a field of view of the diagnostics analysis system; and
an alignment correction system for adjusting the field of view of the diagnostics analysis system responsive to the alignment information.

16. The automated eye examination system as claimed in claim 15, wherein the refractive error composite information includes any of spherical error (defocus) information, cylindrical error (astigmatism) information and cylindrical axis information, and high order aberration errors including any of trefoil error and coma error.

17. The automated eye examination system as claimed in claim 16, wherein the refractive error composite information regarding the eye generally includes spatial mapping information responsive to the refractive error of the inner and peripheral visual field.

18. The automated eye examination system as claimed in claim 17, wherein the spatial mapping is performed via any of non-linear least squares, linear least squares, least absolute residual, bi-square, polynomial regression, or piece-wise linear regression fitting methods.

19. The automated eye examination system as claimed in claim 18, wherein a surface function to fit to map points of the spatial mapping may be a predefined polynomial, a nth-order polynomial, 3D spline, or 3D surface from a lookup table forming a continuous spatial map of spherical error, cylindrical error, cylindrical axis, coma error, trefoil error, or spherical equivalent error information.

20. The automated eye examination system as claimed in claim 15, wherein the refractive error composite information describes components of inner and peripheral refractive errors of the eye's visual field.

21. The automated eye examination system as claimed in claim 15, wherein the alignment correction system includes a tracking mirror.

22. The automated eye examination system as claimed in claim 15, wherein the alignment system further aligns the subject with a field of view of a visual target.

23. The automated eye examination system as claimed in claim 15, wherein the automated eye examination system is provided in a stand-alone kiosk.

24. A method of capturing diagnostic eye information, said method comprising:
directing electromagnetic energy into an eye of a subject;
capturing perception data at each of a plurality of perception units refractive information from the eye responsive to the electromagnetic energy, each perception unit being associated with an associated position in the visual field of the eye;
determining refractive error information associated with each position of each perception unit in the visual field of the eye, and
determining refractive error composite information regarding the eye responsive to the refractive error information associated with each perception unit and independent of a direction of gaze of the eye.

25. The method as claimed in claim 24, wherein at least one energy source is provided among a plurality of energy sources that are positioned at a plurality of locations in the visual field of the eye.

26. The method as claimed in claim 24, wherein the plurality of energy sources are spaced from one another, and each of which is associated with at least one perception unit.

27. The method as claimed in claim 24, wherein each energy source is individually engageable.

28. The method as claimed in claim 24, wherein the refractive error composite information includes any of spherical error (defocus) information, cylindrical error (astigmatism) information and cylindrical axis information, and high order aberration errors including any of trefoil error and coma error.

29. The method as claimed in claim 28, wherein the refractive error composite information regarding the eye generally includes spatial mapping information responsive to the refractive error of the inner and peripheral visual field.

30. The method as claimed in claim 29, wherein the spatial mapping is performed via any of non-linear least squares, linear least squares, least absolute residual, bi-square, polynomial regression, or piece-wise linear regression fitting methods.

31. The method as claimed in claim 29, wherein a surface function to fit to map points of the spatial mapping may be a predefined polynomial, a nth-order polynomial, 3D spline, or 3D surface from a lookup table forming a continuous spatial map of spherical error, cylindrical error, cylindrical axis, coma error, trefoil error, or spherical equivalent error information.

32. The method as claimed in claim 24, wherein the refractive error composite information describes components of inner and peripheral refractive errors of the eye's visual field.
